(19) United States
(12) Patent Application Publication
(10) Pub. No.: US 2025/0297226 A1
(43) Pub. Date: Sep. 25, 2025

(54) CHAOTIC PRINTING FOR THE PRODUCTION OF SCAFFOLDS FOR USE IN CELL CULTURE

(71) Applicants: OHIO STATE INNOVATIION FOUNDATION, Columbus, OH (US); INSTITUTO TECNOLÓGICO Y DE ESTUDIOS SUPERIORES DE MONTERREY, Nuevo León (MX)

(72) Inventors: David DEAN, Columbus, OH (US); Ryan HOOPER, Columbus, OH (US); Grissel TRUJILLO DE SANTIAGO, Monterrey, Nuevo León (MX); Mario Moisés ALVAREZ, Monterrey, Nuevo León (MX); Ciro RODRIGUEZ, Columbus, OH (US)

(21) Appl. No.: 18/861,789

(22) PCT Filed: May 1, 2023
(86) PCT No.: PCT/US23/20586

§ 371 Date: Oct. 30, 2024

Related U.S. Application Data
(60) Provisional application No. 63/337,092, filed on Apr. 30, 2022.

(51) Int. Cl. C12N 5/0775; C12M 1/00; C12M 1/12; C12M 1/42

(52) U.S. Cl. CPC C12N 5/0663; C12M 25/14; C12M 29/10; C12M 35/02; C12M 35/04; C12N 2501/11; C12N 2501/135; C12N 2513/00; C12N 2533/54; C12N 2533/74

(57) ABSTRACT

Provided herein are methods for preparing perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold. In some embodiments, the fugitive ink comprises hydroxyethyl cellulose (HEC). In some embodiments, the the bioink composition and the fugitive ink composition are chaotically printed into a housing comprising a fluid inlet, a fluid outlet, and a path for fluid flow from the fluid inlet to the to generate a microstructured precursor occupying the path for fluid flow. Also provided are scaffolds prepared by these methods as well as modular bioreactors incorporating these scaffolds.

### CROSS-REFERENCE TO RELATED APPLICATIONS

This application claims benefit of U.S. Provisional Application No. 63/337,092, filed Apr. 30, 2022, which is hereby incorporated herein by reference in its entirety.

### BACKGROUND

Cell-based therapies in the clinic are significantly limited by the challenge of quickly and inexpensively producing the required number of cells. Human mesenchymal stem cells (hMSCs) are of particular interest for regenerative medicine due to their ability to differentiate into multiple tissue types including fat, bone, cartilage, and muscle. These cells are also being studied for use in bone marrow transplant and other therapies for hematopoietic cancers. Hundreds of clinical trials involving hMSCs have highlighted the demand for scalable, controlled, and reproducible manufacturing systems that could expand a few million cells from a human donor into hundreds of millions to even billions of hMSCs to be received therapeutically by a single patient. As with the availability of donated tissue and organs, the efficiency and duration of hMSC cell proliferation (expansion) affects the availability of cell-based therapies for patients with cardiovascular, neurodegenerative, musculoskeletal, immunological, and neoplasm disorders. Improvements in hMSC expansion rate and yield would be useful to current therapies, as well as research into new ones.

### SUMMARY

Provided herein are methods for the preparation of perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold. Importantly, these methods can rapidly and efficiently prepare microstructured scaffolds including multiple distinct layers of cells separated by controllable distances. These architectures mimic the biostructures which are involved in tissue and organ development in biological systems.

In certain embodiments, the fugitive ink composition can comprise hydroxyethyl cellulose (HEC).

In certain embodiments, the method can comprise chaotic printing the bioink composition and the fugitive ink composition into a housing comprising a fluid inlet, a fluid outlet, and a path for fluid flow from the fluid inlet to the to generate a microstructured precursor occupying the path for fluid flow.

Also provided are high surface/volume, perfusable microstructured scaffolds for cell culture prepared by the chaotic printing methods described herein. In some embodiments, the

perfusable scaffolds can exhibit an average striation thickness of from 10 nm to 500 μm, a surface-area-to-volume (SAV) of from 400 m⁻¹ to 5000 m⁻¹, a surface density of at least 0.05 m² cm⁻³, or any combination thereof.

Also provided are bioreactors for cell culture/expansion that comprise a plurality of the perfusable scaffolds described herein. The bioreactors can function as incubator-based systems allowing large numbers of cells to be expanded in the smallest possible space. Rather than state of the art indirectly tracked stirring systems, the bioreactor can include highly accurate sensors operatively coupled to each of the plurality of perfusable scaffolds present in the bioreactor. For example, in some embodiments, the perfusable scaffolds can be in the form of rods, fibers, or bundles of fibers. The perfusable scaffolds can be fitted with proximal and distal collars that allow for conditions within each scaffold to be individual perfusable scaffold to be monitored in real time. For example, each collar can incorporate sensors to track environmental gases, nutrient, growth factor delivery, and waste removal. A single input plate can interface with each of the proximal and distal collars. The input plates can apply mechanical (e.g., tension, compression, and/or torsion) and/or electrical stimulation to the proximal and distal collars (and by extension scaffolds) throughout the course of cell culture.

A control system can monitor sensor readings and actuate pumps to alter, for example, media flow rate, levels of bioactive agents, etc. contacting the scaffold. Using this real-time feedback loop, the control system can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth), and/or automatically control media flow rates of available media to ensure cell expansion rates. Unlike non-existent commercial and small scale, home-made systems that deliver non-homogenous mechanical or electrical stimulation, the collared chaotic laminar rod system will allow apply mechanical and electrical stimulation as well as allow automation of cell harvest and storage (freezing). The bioreactor can be housed in a small footprint incubator that facilitates automated and highly accurate control of environmental gases, humidity, and temperature.

### DESCRIPTION OF DRAWINGS

FIG. 1A shows a schematic cross-section of the filament layer for each group in experiment 1.

FIG. 1B illustrates the chaotic printing method used in experiment 1.

FIG. 1C illustrates the chaotic printing method used in experiment 2, resulting in cell-laden hydrogel filaments flush to PP tubes (filament chambers).

FIG. 1D illustrates an example full bioreactor design.

FIGS. 2A-2D show live-cell fluorescent labelling of BM-hMSC-laden hydrogel filaments during experiment 1.

FIG. 2E is a plut showing the cell number calculated from fluorescence intensity readings using PrestoBlue (Invitrogen) viability assay during experiment 1 (**=P<0.01; ****=P<0.0001).

FIG. 3A shows SA filaments flush to edges of filament chambers with layer structures fluorescently labelled.

FIG. 3B shows a filament chamber connected to flow system.

FIG. 3C shows a peristaltic pump and sensor cables leading into incubator, hole sealed with parafilm.

FIG. 3D shows a bioreactor design inside incubator.

FIG. 4 illustrates the live-cell fluorescent labelling of BM-hMSC-laden hydrogel filaments printed with the method used in experiment 2, resulting in thicker diameter and open channels.

FIG. 5 is a schematic diagram of a bioreactor described herein.

### DETAILED DESCRIPTION

The materials, compounds, compositions, systems, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Described herein are improved compositions, systems, and methods that can be used to fabricate scaffolds for use in cell culture. These compositions, systems, and methods can provide cell-laden hydrogel filaments disposed within a chamber or cartridge. These chambers and cartridges can be used within a bioreactor to culture cells and tissues. The chambers and cartridges facilitate the flow of nutrient media in an environment suitable for cell viability and proliferation/expansion (i.e., sterile conditions, 37 degrees Celsius, 5% carbon dioxide).

Many clinical therapies rely on the expansion of a few million cells received from one or more donors to hundreds of millions, if not billions, of cells. One of the more common types of cells that is expanded and then used in cell-based therapies is human Mesenchymal Stem Cells (hMSCs). The systems described herein can culture cells in hydrogel filaments estimated to have 177× higher Surface Areaper-unit-Volume (SAV) than standard cell expansion systems such as microcarrier bioreactors. Since SAV has been found to correlate directly to hMSC expansion rate and total cell yield, the scalable bioreactor designs provided herein should have a significant performance advantage in this regard compared to existing cell expansion bioreactors on the market.

Preliminary experiments have provided proof-of-concept for our bioreactor's ability to expand hMSCs in hydrogel filaments. Those technology-validating results are described in the examples, which detail both methods of producing cell-laden hydrogel filaments that can be placed in a bioreactor, and the bioreactor design itself. These compositions, systems, and methods can further related to the compositions, methods, and systems described in International Publication No. WO 2021/062411, which is incorporated by reference in its entirety.

The methods for producing cell-laden hydrogel filaments that can be connected to the bioreactors described herein can involve a bioprinting technique called chaotic printing. Chaotic printing can produce hydrogel filaments containing alternating layers of two or more materials. When one of these materials is a “fugitive material” or “fugitive ink”, it can be removed from the filament post-chaotic printing. This leaves open channels in between solid, cell-laden hydrogel layers, resulting in an exponential increase in the SAV of interface between cells and their nutrient media.

A printhead design can be used that allows calcium chloride, which solidifies the hydrogel, to be co-axially extruded along with the hydrogel and “fugitive material”. This technology can be applied to production of cell-laden hydrogel filaments to be connected to the bioreactors described herein in the following way: the use of hydroxyethyl cellulose (HEC) was validated as an effective “fugitive material” alongside our hydrogel formulation of sodium alginate (SA, e.g., 2% (w/v)), gelatin methacryloyl (GelMA, e.g., 3% (w/v)), and lithium phenyl-2,4,6-trimethylbenzoylphosphinate (LAP, e.g., 0.067% (w/v)). Two 5-mL syringes containing cell-laden SA-GelMA hydrogel and HEC fugitive material, respectively, can be positioned on a syringe pump. A 5-mL syringe containing CaCl₂ can be fitted to a second syringe pump. The cell-laden hydrogel and fugitive material syringes were connected with rubber tubing to the two inlets on the top of the co-axial printhead, while the CaCl₂ syringe is connected to the inlet near the nozzle tip.

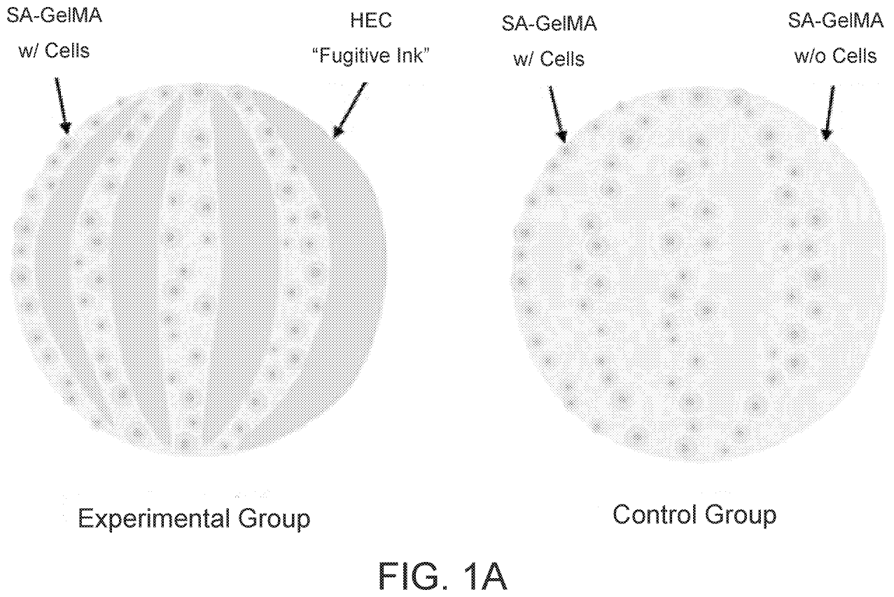

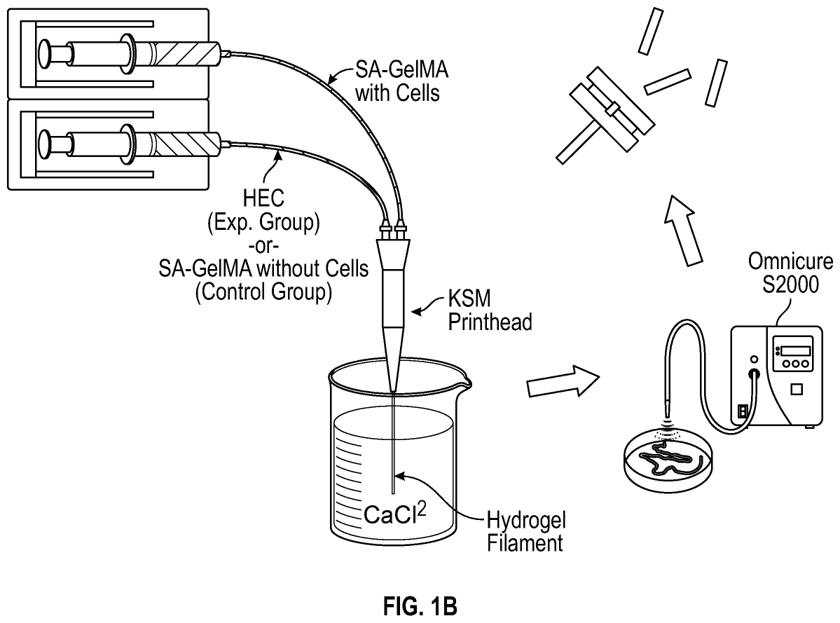

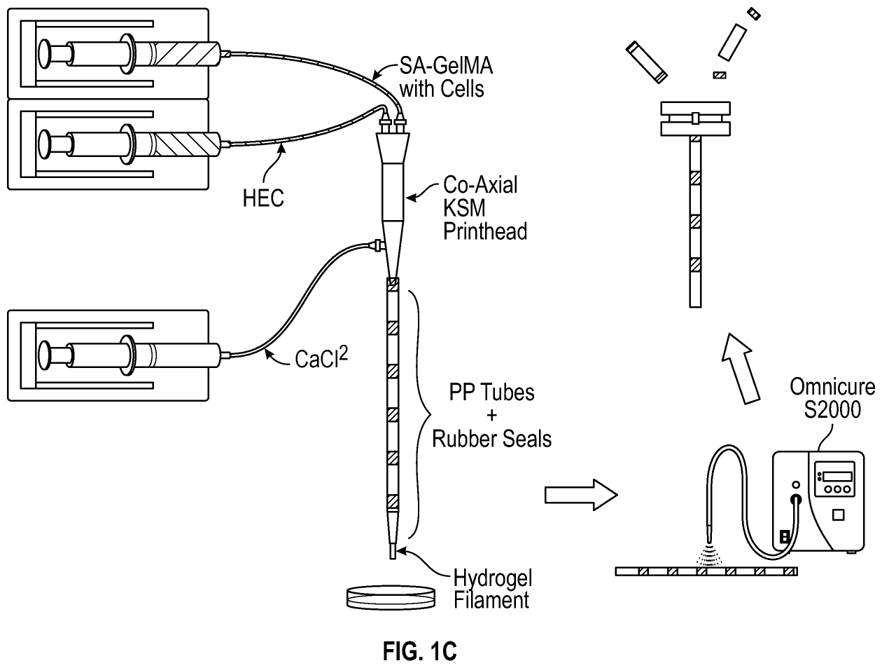

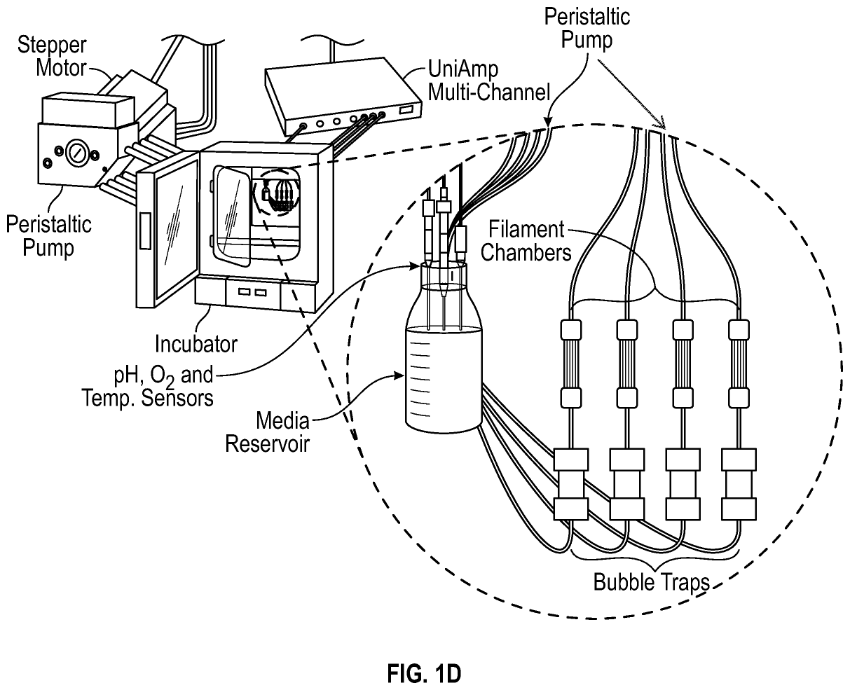

The printhead was fixed to a stand and positioned above a string of 2-cm polypropylene filament chambers connected by rubber seals. Syringe pumps are activated until the cell-laden hydrogel, fugitive material, and CaCl₂ reach their respective inlets. The syringe pump containing the cell-laden hydrogel and fugitive material syringes is then activated to form the chaotic layers by passing the two inks through a series of Kenics Static Mixer (KSM) elements and out the nozzle tip. The CaCl₂ pump is then additionally activated. Solid filament is extruded through the string of connected filament chambers until reaching the end of the tube chain. At this point, the string of chambers is exposed to 365-nm UV light for 30 seconds. A razor blade can be used to cut through the rubber seals connecting each 2-cm PP filament chamber and the remaining rubber pieces are removed. The result of this process is multiple (as many as 16 chambers have been produced from one 2-minute run) PP filament chambers that contain cell-laden hydrogel filaments containing open channels. This process can also allow the filaments to be flush to the inner walls of their respective filament chambers, which is intended to help direct nutrient media flow through the open channels rather than around the filament edges. This process is shown schematically in FIG. 1C.

For example, provided herein are methods for the preparation of perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold.

In some embodiments, chaotic printing can comprise a continuous process. In other embodiments, chaotic printing can comprise a batch process.

Chaotic printing of the bioink composition and the fugitive ink composition can comprise inducing a laminar flow of the bioink composition and the fugitive ink composition through a mixer. The mixer can chaotically mix the bioink composition and the fugitive ink composition, thereby forming lamellar interfaces between the bioink composition and the fugitive ink composition. In some cases, chaotic printing of the bioink composition and the fugitive ink composition can comprise coextruding the bioink composition and the fugitive ink composition through a mixer that chaotically mixes the bioink composition and the fugitive ink composition to form lamellar interfaces between the bioink composition and the fugitive ink composition.

In certain embodiments, the method can comprise chaotic printing the bioink composition and the fugitive ink composition into a housing comprising a fluid inlet, a fluid outlet, and a path for fluid flow from the fluid inlet to the to generate a microstructured precursor occupying the path for fluid flow.

In these embodiments, the mixer can comprise a static mixer, such as a Kenics static mixer (KSM). In some embodiments, the KSM can comprise at least two KSM elements (e.g., at least 3 KSM elements, at least 4 KSM elements, at least 5 KSM elements, at least 6 KSM elements, at least 7 KSM elements, at least 8 KSM elements, or at least 9 KSM elements). In some embodiments, the KSM can comprise 10 KSM elements or less (e.g., 9 KSM elements or less, 8 KSM elements or less, 7 KSM elements or less, 6 KSM elements or less, 5 KSM elements or less, 4 KSM elements or less, or 3 KSM elements or less).

The KSM can comprise a number of KSM elements ranging from any of the minimum values described above to any of the maximum values described above. For example, in some embodiments, the KSM can comprise from 2 to 10 KSM elements (e.g., from 2 to 7 KSM elements, or from 2 to 6 KSM elements).

In some embodiments, chaotic printing of the bioink composition and the fugitive ink composition can comprise coextruding the bioink composition and the fugitive ink composition with a crosslinking agent. By way of example, in some embodiments, the bioink composition can comprise an alginate and the crosslinking agent can comprise a divalent cation. For example, the crosslinking agent can comprise a calcium salt such as calcium chloride.

In some embodiments, chaotic printing the bioink composition and the fugitive ink composition can comprise 3D printing, electrospinning, extrusion, or any combination thereof. In certain embodiments, the chaotic printing process can produce a microstructured filament or fiber. These processes can be used to form a microstructured precursor (and by extension a perfusable scaffold) having a range of 3D shapes.

In certain examples, chaotic printing can comprise extrusion of a microstructured precursor having a variety of 3D shapes (e.g., using processes analogous to those used to produce, for example, pasta noodles of different shapes). For example, chaotic printing can comprise extrusion through a patterned extrusion die to form a microstructured precursor having a desired 3D shape and/or cross-sectional shape.

In certain examples, chaotic printing can comprise of a microstructured precursor in the form of a fiber or filament. In some embodiments, these fibers or filaments can be bundled to form bundles or rods. In some embodiments, these fibers or filaments can be 3D printed or electrospun to form non-woven mats in a variety of 3D shapes.

In some embodiments, the microstructured precursor may be formed into substrate having a desired anatomical shape. For example, the microstructure precursor can be printed, spun, extruded, cast, molded, or a combination thereof to produce a precursor having the three-dimensional shape of, for example, a tissue or organ. In some examples, the precursor can be formed into the shape of a patch for an organ defect (e.g., a segment of cardiac wall, vasculature, or bone), a functioning structure in an organ (e.g., a heart valve), or an entire organ (e.g., a bladder).

Once formed, the microstructured precursor (e.g., the bioink composition present in the microstructured precursor) can be cured. Suitable curing methods can be selected based on the identity of the one or more polymers present in the bioink composition. For example, in some examples, the bioink composition can comprise a polymer (e.g., alginate) which crosslinks upon exposure to a metal cation, such as Ca²⁺. In these examples, curing can comprise contacting the microstructured precursor with an aqueous solution comprising metal cations (e.g., Ca²⁺ ions). In other examples, the bioink composition can comprise one or more polymers that comprise an ethylenically unsaturated moiety. In these examples, curing can comprise exposing the microstructured precursor to UV light. In some embodiments, curing can comprise incubating the microstructured precursor (e.g., for a period of time effective for physical crosslinking of polymer

In certain embodiments, the bioink composition can exhibit a viscosity of less than 1000 cP at 23° C. prior to curing. For example, in some embodiments, the bioink composition can exhibit a viscosity of less than 500 cP, less than 250 cP, or less than 100 cP at 23° C. prior to curing. Upon curing, the bioink composition can increase in viscosity to form a matrix that exhibits a viscosity of at least 25,000 cP at 37° C. (e.g., a viscosity of from 25,000 cP to 100,000 cP at 37° C.)

In certain embodiments, the fugitive ink composition can exhibit a viscosity of less than 1000 cP at 23° C. prior to curing. For example, in some embodiments, the fugitive ink composition can exhibit a viscosity of less than 500 cP, less than 250 cP, or less than 100 cP at 23° C. prior to curing. Upon curing, the fugitive ink composition can retain a viscosity of less than 5,000 cP at 23° C. (e.g., a viscosity of less than 1000 cP, less than 500 cP, less than 250 cP, or less than 100 cP at 23° C.).

Following crosslinking, the fugitive ink can be removed from the cured scaffold precursor. The fugitive ink can be removed by any suitable method. In some embodiments, the fugitive ink can be heated and/or incubated under reduced pressure to drive off the fugitive ink. In other embodiments, the cured scaffold precursor can be immersed in an aqueous solution and/or dialyzed against an aqueous solution to remove the fugitive ink by diffusion. In other embodiments, the cured scaffold precursor can be perfused with an aqueous solution to remove the fugitive ink from within the cured scaffold precursor. Combinations of these methods can also be employed.

In some embodiments, the resulting perfusable scaffolds can exhibit an average striation thickness of at least 10 nm (e.g., at least 25 nm, at least 50 nm, at least 75 nm, at least 100 nm, at least 150 nm, at least 200 nm, at least 250 nm, at least 300 nm, at least 400 nm, at least 500 nm, at least 600 nm, at least 700 nm, at least 750 nm, at least 800 nm, at least 900 nm, at least 1 μm, at least 5 μm, at least 10 μm, at least 20 μm, at least 25 μm, at least 30 μm, at least 40 μm, at least 50 μm, at least 100 μm, at least 200 μm, at least 250 μm, at least 300 μm, or at least 400 μm). In some embodiments, the perfusable scaffolds can exhibit an average striation thickness of 500 μm or less (e.g., 400 μm or less, 300 μm or less, 250 μm or less, 200 μm or less, 100 μm or less, 50 μm or less, 40 μm or less, 30 μm or less, 25 μm or less, 20 μm or less, 10 μm or less, 5 μm or less, 1 μm or less, 900 nm or less, 800 nm or less, 750 nm or less, 700 nm or less, 600 nm or less, 500 nm or less, 400 nm or less, 300 nm or less, 250 nm or less, 200 nm or less, 150 nm or less, 100 nm or less, 75 nm or less, 50 nm or less, or 25 nm or less).

The perfusable scaffolds can exhibit an average striation thickness ranging from any of the minimum values described above to any of the maximum values described above. For example, in some embodiments, the perfusable scaffolds can exhibit an average striation thickness of from 10 nm to 500 μm (e.g., from 10 nm to 50 μm).

In other embodiments, the perfusable scaffolds can include larger striation thicknesses (e.g., striation thicknesses on the millimeter and/or centimeter length scales, such as from 1 mm to 50 cm, or from 1 mm to 10 cm).

In some embodiments, the resulting perfusable scaffolds can exhibit a surface-area-to-volume (SAV) of at least 400 m⁻¹ (e.g., at least 500 m⁻¹, at least 600 m⁻¹, at least 700 m⁻¹, at least 750 m⁻¹, at least 800 m⁻¹, at least 900 m⁻¹, at least 1000 m⁻¹, at least 1250 m⁻¹, at least 1500 m⁻¹, at least 1750 m⁻¹, at least 2000 m⁻¹, at least 2250 m⁻¹, at least 2500 m⁻¹, at least 2750 m⁻¹, at least 3000 m⁻¹, at least 3250 m⁻¹, at least 3500 m⁻¹, at least 3750 m⁻¹, at least 4000 m⁻¹, at least 4250 m⁻¹, at least 4500 m⁻¹, or at least 1750 m⁻¹). In some embodiments, the perfusable scaffolds can exhibit a surface-area-to-volume (SAV) of 5000 m⁻¹ or less (e.g., 4750 m⁻¹ or less, 4500 m⁻¹ or less, 4250 m⁻¹ or less, 4000 m⁻¹ or less, 3750 m⁻¹ or less, 3500 m⁻¹ or less, 3250 m⁻¹ or less, 3000 m⁻¹ or less, 2750 m⁻¹ or less, 2500 m⁻¹ or less, 2250 m⁻¹ or less, 2000 m⁻¹ or less, 1750 m⁻¹ or less, 1500 m⁻¹ or less, 1250 m⁻¹ or less, 1000 m⁻¹ or less, 900 m⁻¹ or less, 800 m⁻¹ or less, 750 m⁻¹ or less, 700 m⁻¹ or less, 600 m⁻¹ or less, or 500 m⁻¹ or less).

The perfusable scaffolds can exhibit a surface-area-to-volume (SAV) ranging from any of the minimum values described above to any of the maximum values described above. For example, in some embodiments, the perfusable scaffolds can exhibit a surface-area-to-volume (SAV) of from 400 m⁻¹ to 5000 m⁻¹.

In some embodiments, the resulting perfusable scaffold can exhibit a surface density of at least 0.05 m² cm⁻³ (at least 0.055 m² cm⁻³, at least 0.06 m² cm⁻³, at least 0.065 m² cm⁻³, at least 0.07 m² cm⁻³, at least 0.075 m² cm⁻³, or more),

### Bioink Compositions

The bioink composition can comprise an aqueous solution comprising one or more polymers (e.g., one or more biopolymers). Following processing, the bioink will form the laminae of the microstructured scaffolds described herein. Accordingly, the one or more polymers can be selected and included in an amount effective such that the polymers form biocompatible laminae suitable to support cell culture upon curing. In some embodiments, the one or more polymers can be biodegradable.

In certain embodiments, the one or more polymers can comprise a hydrogel-forming agent. The term “hydrogel” refers to a broad class of polymeric materials, that may be natural or synthetic, which have an affinity for an aqueous medium, and may absorb large amounts of the aqueous medium, but which do not normally dissolve in the aqueous medium. Generally, a hydrogel may be formed by using at least one, or one or more types of hydrogel-forming agent, and setting or solidifying the one or more types of hydrogel-forming agent in an aqueous medium to form a three-dimensional network, wherein formation of the three-dimensional network may cause the one or more types of hydrogel-forming agent to gel so as to form the hydrogel. The term “hydrogel-forming agent”, also termed herein as “hydrogel precursor”, refers to any chemical compound that may be used to make a hydrogel. The hydrogel-forming agent may comprise a physically cross-linkable polymer, a chemically cross-linkable polymer, or mixtures thereof.

Physical crosslinking may take place via, for example, complexation, hydrogen bonding, desolvation, van der Waals interactions, or ionic bonding. In various embodiments, a hydrogel may be formed by self-assembly of one or more types of hydrogel-forming agents in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains aggregate or coagulate in an aqueous medium to form a three-dimensional network, thereby entrapping molecules of water to form a hydrogel. Examples of physically cross-linkable polymer that may be used include, but are not limited to, gelatin, alginate, pectin, furcellaran, carageenan, chitosan, derivatives thereof, copolymers thereof, and mixtures thereof.

Chemical crosslinking refers to an interconnection between polymer chains via chemical bonding, such as, but not limited to, covalent bonding, ionic bonding, or affinity interactions (e.g. ligand/receptor interactions, antibody/antigen interactions, etc.). Examples of chemically cross-linkable polymer that may be used include, but are not limited to, starch, gellan gum, dextran, hyaluronic acid, poly(ethylene oxides), polyphosphazenes, derivatives thereof, copolymers thereof, and mixtures thereof. Other suitable polymers include polymers (gelatin, cellulose, etc.) functionalized with ethylenically unsaturated moieties (e.g., (meth)acrylate groups). Such polymers may be cross-linked in situ via polymerization of these groups. An example of such a material is gelatin methacrylate (GelMA), which is denatured collagen that is modified with photopolymerizable methacrylate (MA) groups.

Optionally, chemical cross-linking may take place in the presence of a chemical cross-linking agent. The term “chemical cross-linking agent” refers to an agent which induces chemical cross-linking. The chemical cross-linking agent may be any agent that is capable of inducing a chemical bond between adjacent polymeric chains. For example, the chemical cross-linking agent may be a chemical compound. Examples of chemical compounds that may act as cross-linking agent include, but are not limited to, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisacrylamide, N,N′-methylene-bis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, derivatives thereof or mixtures thereof. However, in some embodiments, the hydrogel-forming agents are themselves capable of chemical or physical cross-linking without using a cross-linking agent.

Besides the above-mentioned, the hydrogel-forming agents may be cross-linked using a cross-linking agent in the form of an electromagnetic wave. The cross-linking may be carried out using an electromagnetic wave, such as gamma or ultraviolet radiation, which may cause the polymeric chains to cross-link and form a three-dimensional matrix, thereby entrapping water molecules to form a hydrogel.

In some embodiments, the one or more polymers can comprise a natural polymer. A “natural polymer” refers a polymeric material that may be found in nature. In various embodiments, examples of such natural polymers include polysaccharides, glycosaminoglycans, proteins, and mixtures thereof.

Polysaccharides are carbohydrates which may be hydrolyzed to two or more monosaccharide molecules. They may contain a backbone of repeating carbohydrate i.e. sugar unit. Examples of polysaccharides include, but are not limited to, alginate, agarose, chitosan, dextran, starch, gellan gum, and mixtures thereof. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Peptides, which form building blocks of polypeptides and in turn proteins, generally refer to short chains of amino acids linked by peptide bonds. Typically, peptides comprise amino acid chains of about 2-100, more typically about 4-50, and most commonly about 6-20 amino acids. Polypeptides generally refer to individual straight or branched chain sequences of amino acids that are typically longer than peptides. They usually comprise at least about 20 to 1000 amino acids in length, more typically at least about 100 to 600 amino acids, and frequently at least about 200 to about 500 amino acids. Included are homo-polymers of one specific amino acid, such as for example, poly-lysine. Proteins include single polypeptides as well as complexes of multiple polypeptide chains, which may be the same or different.

Proteins have diverse biological functions and can be classified into five major categories, i.e. structural proteins such as collagen, catalytic proteins such as enzymes, transport proteins such as hemoglobin, regulatory proteins such as hormones, and protective proteins such as antibodies and thrombin. Other examples of proteins include, but are not limited to, fibronectin, gelatin, fibrin, pectins, albumin, ovalbumin, and polyamino acids.

In other embodiments, the one or more polymers can comprise a synthetic polymer. Examples of suitable synthetic polymers include, for example, a polyester such as poly(propylene fumarate) (PPF), polylactic acid (PLA), polyglycolic acid (PGA), poly lactic-co-glycolide (PLGA), polycaprolactone (PCL), polydioxanone (PDS), a polyhydroxyalkanoate (PHA), a polyurethane (PU), copolymers thereof, and blends thereof. Examples of polyhydroxyalkanoates include poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), copolymers thereof, and blends thereof. Other suitable biodegradable synthetic polymers include, for example, polyurethanes. In certain embodiments, the biodegradable synthetic polymer can comprise PGA.

In some embodiments, the one or more polymers can comprise alginate, agarose, or a combination thereof. In some embodiments, the one or more polymers can comprise alginate. The term “alginate” refers to any of the conventional salts of align, which is a polysaccharide of marine algae, and which may be polymerized to form a matrix for use in drug delivery and in tissue engineering due to its biocompatibility, low toxicity, relatively low cost, and simple gelation with divalent cations such as calcium ions (Ca²⁺) and magnesium ions (Mg²⁺). Examples of alginate include sodium alginate which is water soluble, and calcium alginate which is insoluble in water. In some embodiments, the one or more polymers can comprise agarose. Agarose refers to a neutral gelling fraction of a polysaccharide complex extracted from the agarocytes of algae such as a Rhodophycede.

In some embodiments, the one or more polymers can comprise gelatin. The term “gelatin” as used herein refers to protein substances derived from collagen. In the context of this description, “gelatin” also refers to equivalent substances such as synthetic analogues of gelatin (e.g., gelatin methacrylate (GelMA)). Generally, gelatin may be classified as alkaline gelatin, acidic gelatin, or enzymatic gelatin. Alkaline gelatin may be obtained from the treatment of collagen with a base such as sodium hydroxide or calcium hydroxide. Acidic gelatin may be obtained from the treatment of collagen with an acid such as hydrochloric acid. Enzymatic gelatin may be obtained from the treatment of collagen with an enzyme such as hydrolase.

In certain embodiments, the bioink composition can comprise collagen, hyaluronate, fibrin, alginate, agarose, chitosan, gelatin, matrigel, glycosaminoglycans, or a combination thereof.

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the bioink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the bioink composition.

The amount of the one or more polymers present in the bioink composition can be present in an amount ranging from any of the minimum values described above to any of the maximum values described above. For example, in some embodiments, the one or more polymers can be present in the bioink composition in an amount of from 0.5% to 20% by weight, based on the total weight of the bioink composition.

In certain examples, the bioink composition can comprise from 2% by weight to 10% by weight gelatin methacrylate (GelMA) and from 1% by weight to 4% by weight alginate.

In some embodiments, the bioink composition can further comprise a population of cells, one or more bioactive agents, or any combination thereof (as described in more detail below).

The bioink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The bioink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

In some embodiments, the bioink can further include a population of nanoparticles, a population of microparticles, or a combination thereof. In some embodiments, the microparticles and nanoparticles can comprise polymer particles. The polymer particles can be formed from polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some embodiments, one or more bioactive agents (as discussed below) can be conjugated to the surface of the particles. In some embodiments, one or more bioactive agents can be dispersed or encapsulated within the particles. In these embodiments, the particles can provide for the controlled or sustained release of one or more bioactive agents within the laminae over time.

The bioink composition can optionally include one or more additional components, such as a photoinitiator, solvent, surfactant, light attenuator, crosslinker, nutrient, or any combination thereof. In certain examples, the bioink composition can further comprise a photoinitator to facilitate curing.

### Fugitive Ink Compositions

The fugitive ink composition can comprise an aqueous solution comprising one or more polymers which can be readily removed at some point following curing. In some embodiments, the one or more polymers are not crosslinkable or otherwise curable under conditions used to cure the bioink composition. In other embodiments, the one or more polymers can be crosslinkable or otherwise curable, but form a much less robust polymer network upon curing than the cured bioink composition. For example, the one or more polymers present in the fugitive ink composition can initially crosslink or otherwise cure to form fugitive layers following curing. The fugitive layers can then be readily removed while leaving the cured bioink layers (laminae) intact. For example, in some embodiments, the fugitive layer can degrade over time, such that the fugitive layers can be removed some period of time following curing. In other examples, the fugitive layers can decay or dissolve in response to a stimulus (e.g., irradiation with light, heat, contact with an enzyme, or exposure to an acid or base), allowing for removal of the fugitive layers at a desired point following curing.

Examples of suitable polymers include, but are not limited to, polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some examples, the one or more polymers can comprise a polylactide, that is, a lactic acid-based polymer that can be based solely on lactic acid or can be a copolymer based on lactic acid, glycolic acid, and/or caprolactone, which may include small amounts of other comonomers. As used herein, the term “lactic acid” includes the isomers L-lactic acid, D-lactic acid, DL-lactic acid and lactide, while the term “glycolic acid” includes glycolide. Examples include polymers selected from the group consisting of polylactide polymers, commonly referred to as PLA, poly(lactide-co-glycolide) copolymers, commonly referred to as PLGA, and poly(caprolactone-co-lactic acid) (PCL-co-LA), In some examples, the polymer may have a monomer ratio of lactic acid/glycolic acid of from about 100:0 to about 15:85, preferably from about 75:25 to about 30:70, more preferably from about 60:40 to about 40:60, and an especially useful copolymer has a monomer ratio of lactic acid/glycolic acid of about 50:50.

The poly(caprolactone-co-lactic acid) (PCL-co-LA) polymer can have a comonomer ratio of caprolactone/lactic acid of from about 10:90 to about 90:10, from about 35:65 to about 65:35; or from about 25:75 to about 75:25. In certain embodiments, the lactic acid based polymer comprises a blend of about 0% to about 90% caprolactone, about 0% to about 100% lactic acid, and about 0% to about 60% glycolic acid.

The lactic acid-based polymer can have a number average molecular weight of from about 1,000 to about 120,000 (e.g., from about 5,000 to about 50,000, or from about 8,000 to about 30,000), as determined by gel permeation chromatography (GPC). Suitable lactic acid-based polymers are available commercially. For instance, 50:50 lactic acid:glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below.

Examples of other suitable polymers include, but are not limited to, Poly (D,L-lactide) Resomer® L104, PLA-L104, Poly (D,L-lactide-co-glycolide) 50:50 Resomer® RG502, Poly (D,L-lactide-co-glycolide) 50:50 Resomer® RG502H, Poly (D,L-lactide-co-glycolide) 50:50 Resomer® RG503, Poly (D,L-lactide-co-glycolide) 50:50 Resomer® RG506, Poly L-Lactide MW 2,000 (Resomer® L 206, Resomer® L 207, Resomer® L 209, Resomer® L 214); Poly D,L Lactide (Resomer® R 104, Resomer® R. 202, Resomer® R 203, Resomer® R 206, Resomer® R 207, Resomer® R 208); Poly L-Lactide-co-D,L-lactide 90:10 (Resomer® LR 209); Poly glycolide (Resomer® G 205); Poly D,L-lactide-co-glycolide 50:50 (Resomer® RG 504 H, Resomer® RG 504, Resomer® RG 505); Poly D-L-lactide-co-glycolide 75:25 (Resomer® RG 752, Resomer® RG755, Resomer® RG 756); Poly D,L-lactide-co-glycolide 85:15 (Resomer®) RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A (3), MEDISORB® Polymer 5050 DL 3A (3), MEDISORB® Polymer 5050 DL 4A (3)) (Medisorb Technologies International L.P., Cincinati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly s-caprolactone; Poly DL-lactide-co-caprolactone 25:75; and Poly DL-lactide-co-caprolactone 75:25 (Birmingham Polymers, Inc., Birmingham, Ala.).

In some examples, the one or more polymers can comprise a biodegradable, biocompatible poly(alkylene oxide) block copolymer, such as a block copolymer of polyethylene oxide and polypropylene oxide (also referred to as poloxamers). Examples of polyoxyethylene-polyoxypropylene (PEO-PPO) block copolymers include PLURONIC® F127 and F108, which are PEO-PPO block copolymers with molecular weights of 12,600 and 14,600, respectively. Each of these compounds is available from BASF of Mount Olive, N.J. PLURONIC® acid F127 in PBS.

In some examples, the one or more polymers can comprise block polymers such as polyoxyethylene-polyoxypropylene (PEO-PPO) block polymers of the general structure A-B, (A-B)ₙ, A-B-A (e.g., a poloxamer or PLURONIC®), or (A-B-A)ₙ with A being the PEO part and B being the PPO part and n being greater than 1. In other embodiments, the one or more polymers can comprise branched polymers of polyoxyethylene-polyoxypropylene (PEO-PPO) like tetra-functional poloxamines (e.g., a poloxamine or TETRONIC®). For example, the one or more polymers can comprise poloxamer 407, poloxamer 188, poloxamer 234, poloxamer 237, poloxamer 338, poloxamine 1107, poloxamine 1307, or a combination thereof.

Advantageously, poloxamers have surfactant abilities and extremely low toxicity and immunogenic responses. Thus, traces of poloxamers following removal of the fugitive ink can exhibit minimal impact on cells present in the bioink composition and/or cells subsequently seeded into the scaffold.

The average molecular weights of the poloxamers can range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers can contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15 to about 50% of the total mass of the polymer.

In certain embodiments, the fugitive ink composition can comprise hydroxyethyl cellulose (HEC).

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the fugitive ink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the fugitive ink composition.

The amount of the one or more polymers present in the fugitive ink composition can be present in an amount ranging from any of the minimum values described above to any of the maximum values described above. For example, in some embodiments, the one or more polymers can be present in the fugitive ink composition in an amount of from 0.5% to 20% by weight, based on the total weight of the fugitive ink composition.

The fugitive ink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The fugitive ink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

### Cells

Methods can further comprise incorporating a population of cells within the perfusable scaffolds described herein. In some embodiments, methods can further comprise dispersing a population of cells in the bioink composition prior to chaotic printing. As a result, the population of cells can be printed within the perfusable scaffold. Such methods can provide for careful control of cell density and position throughout the perfusable scaffold. For example, by including a population of cells within the bioink composition, scaffolds can be printed including adjacent layers of different types of cells, layers as thin as one cell thick, and/or layers spaced apart from adjacent layers by controllable distances. Such scaffolds mimic environments observed, for example, within an embryo. As such, the scaffolds can provide in improved environment in which to control, for example, cellular differentiation. In certain embodiments, two or more distinct populations of cells (e.g., two different types of cells can be printed within the perfusable scaffold. In other embodiments, the perfusable scaffold can be seeded with a population of cells following printing (e.g., by profusion with a fluid containing a population of cells dispersed therein).

The population of cells can include any desired population of viable cells. The viable cells may include any mammalian cell type selected from cells that make up the mammalian body, including germ cells, somatic cells, and stem cells. Depending on the type of cell, cells that make up the mammalian body can be derived from one of the three primary germ cell layers in the very early embryo: endoderm, ectoderm or mesoderm. The term “germ cells” refers to any line of cells that give rise to gametes (eggs and sperm). The term “somatic cells” refers to any biological cells forming the body of a multicellular organism; any cell other than a gamete, germ cell, gametocyte or undifferentiated stem cell.

For example, in mammals, somatic cells make up all the internal organs, skin, bones, blood and connective tissue. As such, a cell may include any somatic cell isolated from mammalian tissue, including organs, skin, bones, blood and connective tissue (i.e., stromal cells). Examples of somatic cells include fibroblasts, chondrocytes, osteoblasts, tendon cells, mast cells, wandering cells, immune cells, pericytes, inflammatory cells, endothelial cells, myocytes (cardiac, skeletal and smooth muscle cells), adipocytes (i.e., lipocytes or fat cells), parenchyma cells (neurons and glial cells, nephron cells, hepatocytes, pancreatic cells, lung parenchyma cells) and non-parenchymal cells (e.g., sinusoidal hepatic endothelial cells, Kupffer cells and hepatic stellate cells). The term “stem cells” refers to cells that have the ability to divide for indefinite periods and to give rise to virtually all of the tissues of the mammalian body, including specialized cells. The stem cells include pluripotent cells, which upon undergoing further specialization become multipotent progenitor cells that can give rise to functional or somatic cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue.

In some examples, the cells can comprise pluripotent stem cells, multipotent stem cells, progenitor cells, terminally differentiated cells, endothelial cells, endothelial progenitor cells, immortalized cell lines, primary cells, or any combination thereof.

When added to the bioink composition prior to chaotic printing, the concentration of cells may vary depending on the composition and quantity of the bioink composition. In embodiments, the concentration of cells in the bioink composition may be in the range from about 1×10³ cells m⁻¹ to about 1×10¹⁰ cells m⁻¹ of bioink composition, such as about 1×10³ cells m⁻¹ to about 1×10⁷ cells m⁻¹, about 1×10⁵ cells m⁻¹ to about 1×10⁷ cells m⁻¹, about 1×10⁵ cells m⁻¹ to about 1×10¹⁰ cells m⁻¹, about 1×10⁷ cells m⁻¹ to about 1×10¹⁰ cells m⁻¹, about 1×10⁵ cells m⁻¹, about 1×10⁶ cells m⁻¹, about 1×10⁷ cells m⁻¹, or about 1×10⁸ cells m⁻¹.

### Bioactive Agents

In some embodiments, the bioink composition can further include one or more bioactive agents. These bioactive agents can ultimately be incorporated into the laminae of the scaffolds therein. In some embodiments, the bioactive agents can be dissolved or dispersed in the bioink composition. In some embodiments, the bioactive agents can be bioconjugated to one or more polymers present in the bioink composition. In other embodiments, the perfusable scaffold can be treated with one or more bioactive agents following synthesis (e.g., by perfusing the scaffold with a solution or suspension comprising one or more bioactive agents). Such an approach may also be used to generate gradients of cues within the scaffold. Cells respond to gradients of fixed and diffusible chemical cues during development, wound healing and inflammatory responses that can direct cell migration, proliferation and differentiation.

As used herein, “bioactive agents” refers to any chemical substances that have an effect in a biological system, whether such system is in vitro, in vivo, or in situ. Examples of classes of bioactive agents include, but are not limited to growth factors, cytokines, antiseptics, antibiotics, anti-inflammatory agents, chemotherapeutic agents, clotting agents, metabolites, chemoattractants, hormones, steroids, morphogens, growth inhibitors, other drugs, or cell attachment molecules.

The term “growth factors” refers to factors affecting the function of cells such as osteogenic cells, fibroblasts, neural cells, endothelial cells, epithelial cells, keratinocytes, chondrocytes, myocytes, cells from joint ligaments, and cells from the nucleus pulposis. Examples of growth factors include platelet derived growth factors (PDGF), the transforming growth factors (TGF-beta), insulin-like growth factors (IGFs), fibroblast growth factors (FGFs), VEGF, EGF, and the bone morphogenetic proteins (BMPs).

The term “cytokines” refers to peptide protein mediators that are produced by immune cells to modulate cellular functions. Examples of cytokines include, but are not limited to, interleukin-1β (IL-1β), interleukin-6 (IL-6), and tumor necrosis factor-α (TNFα).

The term “antiseptics” refers to a chemical agent that inhibits growth of disease-carrying microorganisms. Examples of antiseptics include peroxides, C6-C14 alkyl carboxylic acids and alkyl ester carboxylic acids, antimicrobial natural oils, antimicrobial metals and metal salts such as silver, copper, zinc and their salts.

The term “antibiotic” includes bactericidal, fungicidal, and infection-preventing drugs which are substantially water-soluble such as, for example, gentamicin, vancomycin, penicillin, and cephalosporins. An antibiotic can be added, for example, for selection of the cells or to prevent bacterial growth.

The term “anti-inflammatory agent” refers to any agent possessing the ability to reduce or eliminate cerebral edema (fluid accumulation) or cerebral ischemia, and can include examples such as free radical scavengers and antioxidants, non steroidal anti-inflammatory drugs, steroidal anti-inflammatory agents, stress proteins, or NMDA antagonists.

The term “chemotherapeutic agents” refer to any natural or synthetic molecules that are effective against one or more forms of cancer, and may include molecules that are cytotoxic (anti-cancer agent), simulate the immune system (immune stimulator), or molecules that modulate or inhibit angiogenesis. Examples of chemotherapeutic agents include alkylating agents, antimetabolites, taxanes, cytotoxics, and cryoprotectant adjuvants.

The term “clotting agent” refers to refers to any molecule or compound that promotes the clotting of blood. Examples of clotting agents include a thrombin agent, which is commonly used as a topical treatment by vascular surgeons to stop surface bleeding after a large surface incision is made in the body, heparin, warfarin, and coumarin derivatives.

The term “metabolite” refers to an intermediate or a product derived from enzymatic conversion of a substrate administered to a subject, the conversion occurring as part of a metabolic process of the subject. Examples of metabolite include glucose, carbohydrates, amino acids and lipids.

The term “chemoattractants” refers to a substance that elicits accumulation of cells, such as chemokines, monocyte chemoattractant protein-1, and galectin-3.

The term “hormone” refers to trace substances produced by various endocrine glands which serve as chemical messengers carried by the blood to various target organs, where they regulate a variety of physiological and metabolic activities in vertebrates. Examples of hormones include steroidal estrogens, progestins, androgens, and the progestational hormone progesterone. Steroids may also be classified as lipids. Naturally occurring steroids are hormones that are important regulators of animal development and metabolism at very low concentrations. Examples of steroids include cholesterol, cortisone, and derivatives of estrogens and progesterones.

The term “cell attachment molecules” as used herein includes, but is not limited to, fibronectin, vitronectin, collagen type I, osteopontin, bone sialoprotein thrombospondin, and fibrinogen. Such molecules are important in the attachment of anchorage-dependent cells.

### Bioreactors

Also provided are bioreactors for cell culture/expansion. The bioreactors can be configured to maintain and incubate a plurality of the perfusable scaffolds described herein under conditions suitable for growth of the cells. That is, the bioreactor can house the perfusable scaffolds described herein under adequate environmental conditions to permit a population of cells present therein to survive, proliferate, differentiate and/or express certain products. “Cell growth” means that the cells survive and preferably, though not exclusively, divide and multiply. In some embodiments, the perfusable scaffolds, the bioreactor, or a combination thereof may be adapted to induce tissue generation.

The bioreactors described herein can function as incubator-based systems allowing large numbers of cells to be expanded in the smallest possible space. Rather than state of the art indirectly tracked stirring systems, the bioreactor can include highly accurate sensors operatively coupled to each of the plurality of perfusable scaffolds present in the bioreactor. For example, in some embodiments, the perfusable scaffolds can be in the form of rods, fibers, or bundles of fibers. The perfusable scaffolds can be fitted with proximal and distal collars that allow for conditions within each scaffold to be individual perfusable scaffold to be monitored in real time. For example, each collar can incorporate sensors to track environmental gases, nutrient, growth factor delivery, and waste removal. A single input plate can interface with each of the proximal and distal collars. The input plates can apply mechanical (e.g., tension, compression, and/or torsion) and/or electrical stimulation to the proximal and distal collars (and by extension scaffolds) throughout the course of cell culture.

A control system can monitor sensor readings and actuate pumps to alter, for example, media flow rate, levels of bioactive agents, etc. contacting the scaffold. Using this real-time feedback loop, the control system can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth), and/or automatically control media flow rates of available media to ensure cell expansion rates. Unlike non-existent commercial and small scale, home-made systems that deliver non-homogenous mechanical or electrical stimulation, the collared chaotic laminar rod system will allow apply mechanical and electrical stimulation as well as allow automation of cell harvest and storage (freezing). The bioreactor can be housed in a small footprint incubator that facilitates automated and highly accurate control of environmental gases, humidity, and temperature.

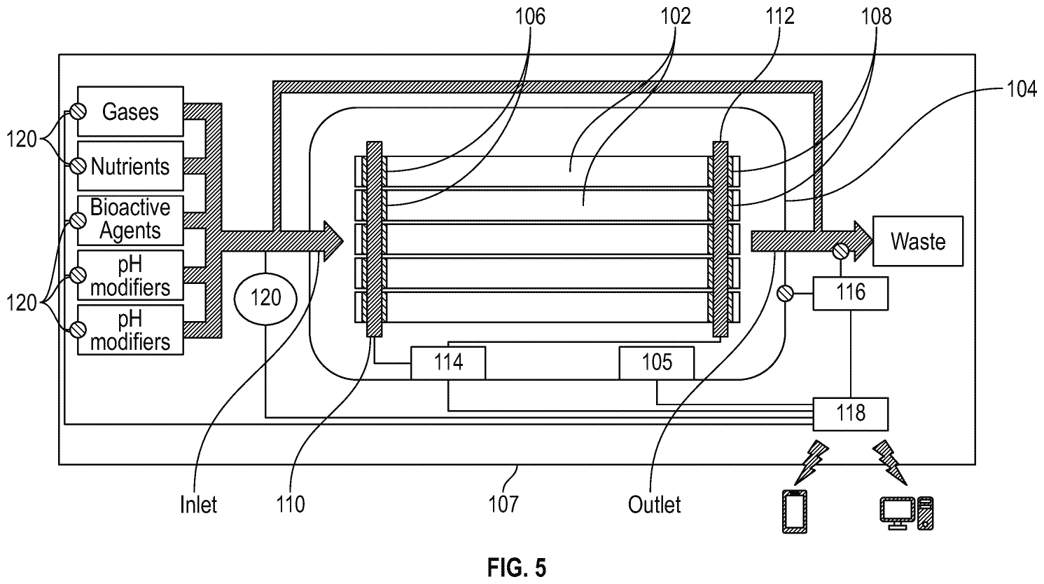

Referring to FIG. 5, in one embodiment the bioreactor can comprise a plurality of perfusable scaffolds (102), each prepared by the methods described herein. Each of the plurality of the perfusable scaffolds is in the form of a rod, fiber, or bundle of fibers. A housing (104) can enclose the plurality of perfusable scaffolds. Heating and cooling elements (105) can be positioned within the housing to allow for thermostatic control of the interior of the housing (e.g., to maintain physiological temperature within the housing). Likewise, sensors (116) can be positioned to monitor temperature within the housing.

Each of the plurality of perfusable scaffolds (102) is operatively coupled to a proximal collar (106) and a distal collar (108). A first single input plate (110) can be operatively coupled to each of the proximal collars (106), and a second single input plate (112) can be operatively coupled to each of the distal collars (108). The first single input plate (110) and the second single input plate (112) can be operatively coupled to one or more actuators (114) that can apply mechanical stimulation to the plurality of perfusable scaffolds, electrical stimulation to the plurality of perfusable scaffolds, or a combination thereof. Sensors (116) can also be operatively connected to the outlet to monitor the composition of fluid flowing from the outlet (e.g., environmental gases, nutrients, growth factor delivery, concentration of biomarkers, concentration of bioactive agents, pH and waste removal) in real time.

In some embodiments, some or all of the system can be positioned within an incubator (107). A control system (118) can monitor sensor readings and actuate pumps (120) to alter, for example, media flow rate, levels of bioactive agents, etc. contacting the scaffold. Using this real-time feedback loop, the control system (118) can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth). These aspects of the bioreactor can operate as a pH monitoring and control system, a temperature monitoring and control system, an O₂ monitoring and control system, a CO₂ monitoring and control system, a glucose monitoring and control system, a lactate monitoring and control system, a fluid flow monitoring and control system, or any combination thereof (depending on which components are individually sensed by sensors in the bioreactor, present in reservoirs, and coupled to pumps operated by the control system).

Current lab-based use of non-GMP cells in standard footprint incubators can expand about 50 flasks of 1 million to 100 million cells (i.e., 5 billion cells). Whole room systems are available to expand up to 25-30 billion cells. The bioreactors described herein will accomplish this in a bench-top incubator. For comparison, example densities of different bioreactor systems are compared in the table below.

Cells Per Milliliter: Conventional Bioreactors Vs. Bioreactors Described Herein.

| Bioreactor | cells/mL* |
|---|---|
| In a T-175 cell culture bottle | 7.37 × 10⁵ |
| In a Petri Dish (9 cm diameter) | 2.72 × 10⁶ |
| Hollow-fiber bioreactors | 3.17 × 10⁶ |
| Spinner with Microcarriers | 7.85 × 10⁶ |
| In a Fedbatch bioreactor (suspension culture) | 3.85 × 10⁷ |
| Bioreactors described herein | 1.25 × 10⁸ |

*Calculated assuming a cell density of 2.72 × 10⁶ cell/cm²

Moreover, scaffold modularity can improve expansion speed, require less handling or space for freezing, and the use of standard incubators with direct sensing/controls will reduce cost at least 10×.

By way of non-limiting illustration, examples of certain embodiments of the present disclosure are given below.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Chaotic Printing of Hydrogel Carriers for Human Mesenchymal Stem Cell Expansion

Increasing expansion rate in cell manufacturing systems would allow faster delivery of life-saving treatments to a greater number of patients. Chaotic printing is a biofabrication technology that can improve cell expansion capabilities by producing hydrogel filaments layered with open channels, resulting in significantly higher surface area-per-unit-volume of interface between cells and nutrient media compared to existing systems. In this study, we chaotically printed hydrogel filaments laden with clinically-relevant, bone marrow-derived human mesenchymal stem cells. After the first week of a 21-day expansion period, cell-laden hydrogel filaments containing open channels had an optimal expansion rate and 2.1× as many cells as filaments without channels. We then validated a novel bioreactor design for expanding cells under flowing nutrient media conditions.

### INTRODUCTION

hMSC expansion rate and total yield are known to correlate directly with the surface area-per-unit-volume (SAV) of the cell expansion system. Chaotic printing is a novel, patent-pending, high-resolution (as small as ˜10 nm) biofabrication technology that can produce layered hydrogel filaments estimated to have 177× higher SAV than standard cell expansion systems such as microcarrier bioreactors. Using a Kenics Static Mixer (KSM), cell-laden polymer (bioink) layers can be interspersed with “fugitive” polymer (fugitive ink) layers in chaotically printed filaments. Once the fugitive ink clears, newly opened channels are in direct contact with cell-laden layers, resulting in an exponentially larger interface between nutrient media and cells compared to standard cell expansion systems. Thus, the high SAV of this interface may allow increased cell expansion by increasing nutrient delivery to each cell and expediting waste product removal. We have already demonstrated the viability of muscular murine cells (C2C12 cell line) in chaotically printed hydrogel filaments comprised of sodium alginate (SA) and gelatin methacryloyl (GelMA). We present here the initial results from our study of chaotic printing for the expansion of clinically-relevant hMSCs. In this example, we first observed the expansion of bone marrow-derived hMSCs (BM-hMSCs) in chaotically printed SA-GelMA hydrogel filaments over a 21-day culture period. The expansion rate of BM-hMSC-laden filaments with open channels was compared to BM-hMSC-laden solid control filaments (i.e., no fugitive ink layers). We then observed expansion and yield of chaotically-printed, BM-hMSC-laden hydrogel filaments within a bioreactor design.

### Materials and Methods

Printhead Fabrication. The KSM printheads used for chaotic printing in this example were fabricated with a digital light processing (DLP) projector-based Perfactory 3 Mini Multi Lens 3D printer from EnvisionTEC (Dearborn, MI). E-Shell 300 resin (EnvisionTEC) was chosen as a bioinert printing material compatible with BM-hMSCs. STL files of the printhead designs were imported into Perfactory RP software to generate supports along with a 3D printable (i.e., slice-by-slice) “build” file. 3D printed KSM printhead parts were cleaned with acetone, isopropanol, ethanol, and deionized water and supports were removed. Cleaned parts were then post-cured for 1 hour in a 3D Systems Procure 350 (Rock Hill, SC) UV light box.

Ink Synthesis. Ink formulations previously proven to successfully produce layered filaments and be non-cytotoxic for C2C12 cells were translated to this BM-hMSC experiment. 2% (w/v) SA, 3% (w/v) GelMA, and 0.067% (w/v) lithium phenyl-2,4,6-trimethylbenzoylphosphinate (LAP) were constituted in sterile Dulbecco's phosphate-buffered saline (DPBS) to serve as a base for the cell-laden bioink, while 0.4% (w/v) hydroxyethyl cellulose (HEC) was constituted in sterile deionized water to form the fugitive ink. SA, GelMA, LAP, and HEC powders were sterilized with UV light before being added to their respective solutions, which were then stirred and heated at 70° C. for 15 minutes in a sterile environment. BM-hMSCs were pelleted before being homogenized in the SA-GelMA bioink at a density of 2 million cells/mL.

Cell Preparation. The BM-hMSCs used in this example were purchased from RoosterBio (Frederick, MD) at passage 3. Cells were thawed and incubated at 37° C. and 5% CO₂ in RoosterBio RoosterBasal-MSC media that was supplemented with 10% v/v fetal bovine serum (FBS) and 1% v/v penicillin-streptomycin (P/S) from Gibco (Grand Island, NY). Once the cells reached approximately 70-80% confluence, they were washed 3 times with phosphate-buffered saline (PBS) (Gibco) and detached with TrypLE (Gibco). The resulting cell suspension was pelleted down by centrifuging at 1200 rpm for 5 minutes and aspirating the remaining media. Cells were then reconstituted in fresh media. 10 μL of this cell suspension was combined with 80 μL of PBS and 10 μL of TrypanBlue (Gibco) before being counted with a hemocytometer.

Experiment 1—Channeled Filaments vs. Solid Filaments. In experiment 1, hydrogel filaments were chaotically printed directly into a beaker containing calcium chloride (CaCl₂)). See FIG. 1B. Two 5-mL syringes were filled with BM-hMSC-laden SA-GelMA bioink and HEC fugitive ink, respectively, and positioned on a Chemyx Fusion 200 syringe pump (Stafford, TX) to be extruded simultaneously through a 1-mm-diameter nozzle at a 1.5 mL/min flow rate. The syringes were connected by Cole-Palmer (Vernon Hills, IL) 1/16″ rubber tubing to each inlet on the KSM printhead. The syringe pump was used to extrude the bioink and fugitive ink through the printhead, which was pointed upward to avoid air bubbles. Extrusion was momentarily halted once the combined ink reached the tip of the printhead nozzle and all extruded ink was wiped from the nozzle. With the printhead nozzle submerged in the beaker containing CaCl₂), extrusion was re-initiated so the SA component of the combined ink would crosslink and further cure (i.e., stiffen) upon contact with CaCl₂. Clogging was avoided by continuously moving the printhead across the beaker during extrusion. To crosslink the GelMA component, filaments were submerged in PBS in petri dishes and exposed to 365-om UV light for 30 seconds. This process produced filaments with alternating BM-hMSC-laden hydrogel layers and open channels for the experimental group. To create filaments with alternating layers of cell-laden hydrogel and no-cell hydrogel for the control group, these protocols were conducted with two 5-mL syringes containing cell-laden SA-GelMA hydrogel and no-cell SA-GelMA hydrogel, respectively (note: no fugitive hydrogel was included in this group). See FIG. 1A. All the resulting filaments were cut into 1-cm segments with a razor blade and placed in ultra-low-attachment, 24-well plates from Corning (Corning, NY). Samples were organized in groups of 6 for cell number and expansion measurements and groups of 3 for fluorescence imaging, respectively. Each well was filled with 1 mL of the following cell “proliferation media”: RoosterBasal-MSC media (RoosterBio) supplemented with 2% (v/v) RoosterBooster-MSC-XF (RoosterBio), 0.05% (v/v) of 10 ng/μL fibroblast growth factor (FGF), 0.2% (v/v) of 10 ng/μL epidermal growth factor (EGF), and 0.4% (v/v) of 10 ng/μL platelet derived growth factor (PDGF). The well plates were incubated for 21 days at 37° C. and 5% CO₂. Media was replaced every 48 hours for the 21-day duration, with cell number and expansion measurements and fluorescence imaging being conducted on days 1, 7, 14, and 21 of cell expansion.

Experiment 2—Flow Conditions vs. Static Conditions. In experiment 2, a printhead design was used that allows CaCl₂) to be co-axially extruded alongside the combined bioink and fugitive ink. See FIG. 1C. This allows solid filaments to be extruded directly from a 1.75-mm-diameter nozzle into polypropylene (PP) bioreactor tubes (filament chambers) without requiring CaCl₂) submersion. This printing method was first attempted with 2% SA and 0.6% HEC to ensure that desired filament structures were produced before attempting to print with cells. After this initial validation step, two 5-mL syringes containing cell-laden SA-GelMA bioink and HEC fugitive ink, respectively, were positioned on a syringe pump as in experiment 1. A 5-mL syringe containing CaCl₂ was fit to a second syringe pump. The bioink and fugitive ink syringes were connected with rubber tubing to the two inlets on the top of the KSM printhead, while the CaCl₂ syringe was connected to the inlet near the nozzle tip. The printhead was fixed to a stand and positioned above a string of 2-cm filament chambers connected by rubber seals. Syringe pumps were activated until the bioink, fugitive ink, and CaCl₂) reached their respective inlets. The syringe pump containing the bioink and fugitive ink syringes was then activated to form the chaotic layers by passing the two inks through a series of KSM elements and out the nozzle tip. The CaCl₂) pump was then additionally activated. Solid filament was extruded through the string of connected filament chambers until reaching the end of the tube chain. At this point, the string of chambers was exposed to 365-nm UV light for 30 seconds. A razor blade was used to cut through the rubber seals connecting each 2-cm filament chamber and the remaining rubber pieces were removed. Eight of the 2-cm filament chambers were situated in a bioreactor segment that could be connected to a syringe and filled with 0.5 mL of media at regular intervals to commence media infusion and waste product extrusion. Half of these samples had their media replaced using a syringe twice per day, while the other half was incubated without media replacement. 3 more samples were added to an ultra-low-attachment, 6-well plate (Corning) to serve as a control group cultured in 7 mL of static media. All samples were incubated at 37° C. and 5% CO₂ for 7 days using the proliferation media formulation described above. Cell number and expansion data was collected in the following manner: On day 0 (directly post-printing) for all groups, day 7 for the flow tubing group and static plate group, and days 1, 2, 3, and 4 for the static tubing group.

Determining Cell Number and Expansion Rate. PrestoBlue HS Cell Viability Reagent from Invitrogen (Waltham, MA) was used to track and measure cell number in this example as follows: Each sample was incubated at 37° C. and 5% CO₂ for 45 minutes in 1 mL of a 1:10 PrestoBlue-to-proliferation media solution. Four 200-μL aliquots were taken from each sample and pipetted into a 96-well plate. Bottom-read fluorescence intensity was measured from the 96-well plate at 560-nm excitation, 590-nm emission, and 10-nm bandwidth using a Cytation 5 Multi-Mode Reader from BioTek (Santa Clara, California). Relative fluorescence units were converted to cell number by creating a standard curve. BM-hMSCs were suspended in wells at graded concentrations of 250K, 125K, 62.5K, 31.25K, 15.625K, and 0 cells/0.5 mL in triplicate, respectively. PrestoBlue reagent was added to each well to produce a 1:10 PrestoBlue-to-proliferation media ratio in 1 mL of solution. The same protocol described above was used to produce relative fluorescence units for known cell concentrations. A linear trendline was fit to a plot of cell number to relative fluorescence units on the x and y axes, respectively, with an R² value of 0.9996. The equation for this trendline was then used to convert relative fluorescence units to cell number in the example.

Determining Cell Viability, Morphology, and Orientation. Live/Dead Viability Assay from Invitrogen (Waltham, MA) was used to visualize the morphology and orientation of BM-hMSCs within chaotically printed hydrogel filament layers. Filament samples were submerged in a solution of 2 μM calcein AM and 4 AM ethidium homodimer-1 (EthD-1) constituted in DPBS for 45 minutes at room temperature. The samples were then washed three times with PBS and imaged using a Cytation 5 Multi-Mode Reader (BioTek).

Bioreactor Set-Up and Function. A bioreactor was constructed. See FIG. 1D. First, 2-cm sections were cut from 3.5-mm-diameter PP insulin syringes to serve as the filament chambers that connect to the flow circuit via 1/16″ rubber tubing. To prevent hydrogel from being driven out of the filament chambers when media began to flow through the bioreactor circuit, stainless steel fine mesh screens were cut and fit into each rubber end piece attached to the filament chambers. A pH electrode, 02 optode, and thermosensor, all from Unisense (Aarhus, Denmark), were situated in the media reservoir and sealed with parafilm. Flow tubing and sensor wires were fed out of a hole in the back of the incubator, which was sealed with parafilm, so that the peristaltic pump and sensor amplifier could reside outside of the incubator. Chaotically printed hydrogel filaments of 2% SA with green fluorescent particles were printed flush to filament chambers and attached to the pump system as a no-cell validation of function. A flow rate of 0.1 mL/min was applied to hydrogel filaments housed in the filament chambers for no longer than 5 minutes at a time. To observe whether the hydrogel filament could withstand flow without being damaged or displaced, flow was applied twice a day for two days in a row.

Filament Measurements. Hydrogel filament diameters were calculated by tracing fluorescence microscopy images in ImageJ software (NIH).

Statistical Analysis. PrestoBlue cell number data was averaged and compared across groups using a paired, two-tailed T-test. A p-value less than 0.05 was considered statistically significant. Standard deviation was also calculated, and 95% confidence intervals were generated for each group.

### Results and Discussion

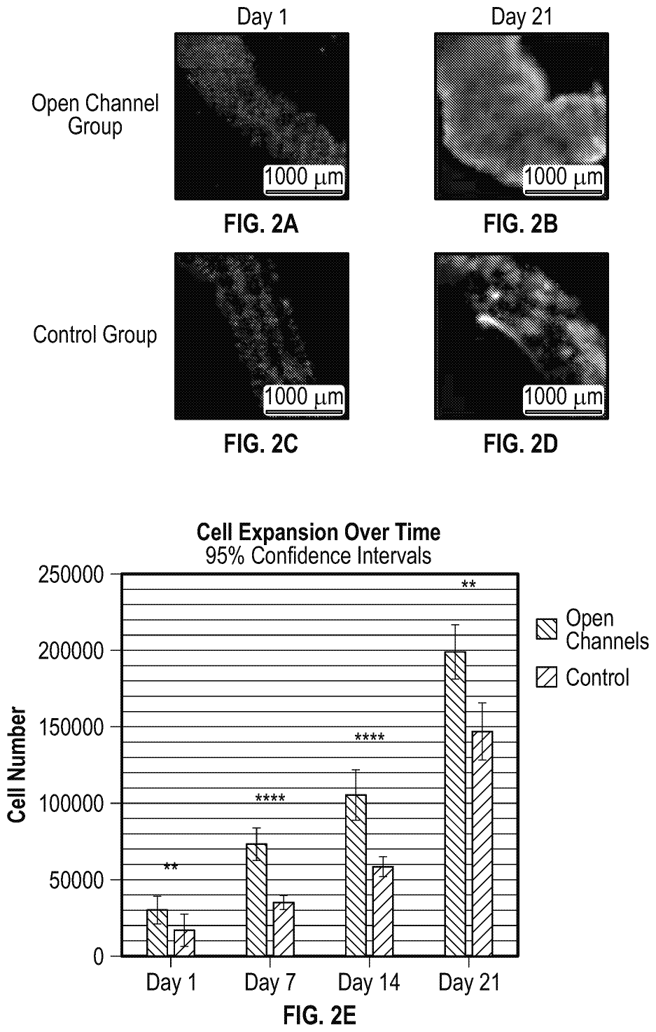

Cell Viability and Expansion in Experiment 1. Live-cell fluorescent images showed both the open channel experimental group and the control group BM-hMSC-laden hydrogel filaments maintaining viability and expansion throughout a 21-day cell expansion period (FIGS. 2A-2D). This validated the lack of cytotoxicity of our SA-GelMA bioink with BM-hMSCs and the overall chaotic printing system. From fluorescent images, open channel hydrogel filaments had a higher density of cells than the control group filaments by day 21. Cells in both groups had an elongate morphology by day 21. Control group cells appeared to elongate parallel to the layer structure (FIG. 2D), adding to evidence that chaotically printed hydrogel structures can induce cell elongation and alignment. Our PrestoBlue data determined that the BM-hMSC-laden hydrogel filaments with open channels had significantly more cells than the control filaments on days 1, 7, 14, and 21 of expansion (FIG. 2E). This confirms that the presence of open channels and the resultant increase in SAV has a positive impact on cell expansion rate and total yield in chaotically printed, BM-hMSC-laden hydrogel filaments. The highest average expansion rate in the open channel group occurred during the first week of culture with a 240% (2.4×) increase in cell number over that span. The open channel group also reached the highest percent increase over the control group on day 7 with 210% (2.1×) as many cells per filament. These numbers highlight the first week of culturing BM-hMSCs in chaotically printed hydrogel filaments as a period of optimal cell expansion.

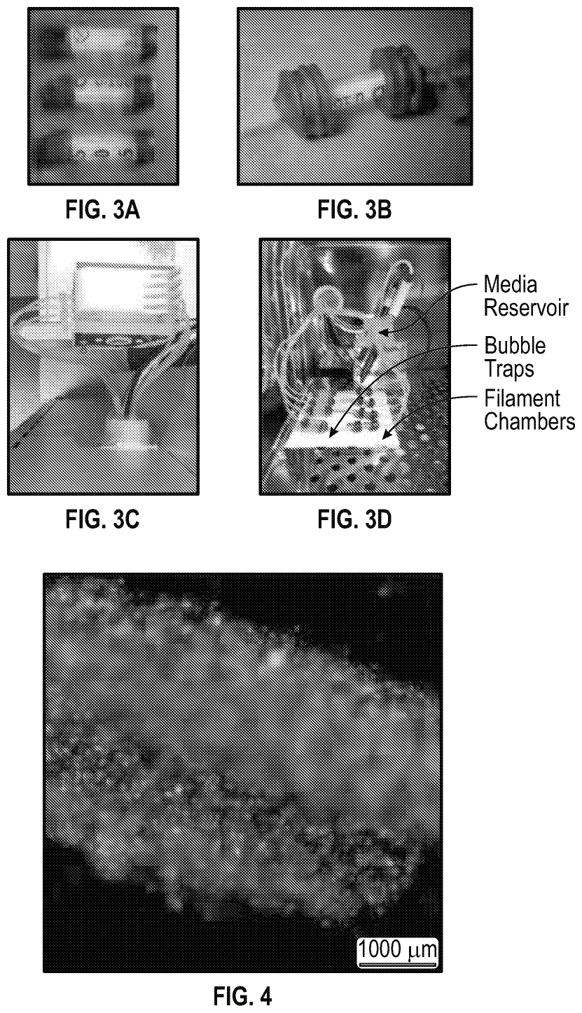

Filament-Filled Tube Fabrication Technique Validation. Initial tests with 2% (w/v) SA and 0.6% (w/v) HEC helped ensure the effectiveness of chaotic printing directly into serially connected 2-cm filament chambers with co-axial CaCl₂ flow. Chaotically printed hydrogel filament travelled smoothly from the printhead through the filament chambers. The resulting tubes filled with hydrogel filament were easily separated by cutting through the rubber connecting pieces with a razor blade and removing them. Fluorescent particles embedded in the SA hydrogel allowed for clear visualization of layer structures under UV light. FIG. 3A demonstrates the structural consistency of filaments produced with extrusion into serially connected chambers and subsequent sectioning. The filaments appear to be flush with the inner wall of the chambers. This result was intended to direct flow through the filament channels rather than around the outer surface of the filaments. This fabrication method allows for at least 16 2-cm chambers of filament to be produced from a single continuous extrusion period. It is important to note that this does not require repeated starting and stopping of flow through the co-axial printhead to produce each filament chamber. Repeated stopping and starting of flow with co-axial KSM printheads was found to quickly cause significant blockage of flow through the printhead nozzle. A single 2-cm hydrogel-filled filament chamber connected to a flow circuit is shown in FIG. 3B. Water flowed through the filament chamber at 0.1 mL/min and did not damage or displace the filament, validating the effectiveness of the metal meshes incorporated into each end piece.

Full Bioreactor Validation. Flow was successfully established from a peristaltic pump outside our incubator to four bioreactor circuits on the inside (FIGS. 3C-3D). The circuits were built with as few level changes as possible to minimize gravitational effects on flow. Bioreactor pH, O₂ concentration, and temperature were measured inside a media reservoir connected to the bioreactor circuits within the incubator. Cell-laden filaments within filament chambers maintained viability after being subjected to flow at 0.1 mL/min for 5 minutes (FIG. 4). The average diameter of filaments printed into filament chambers using experiment 2 methods (FIG. 2C) was approximately 2.5 mm. The largest diameter filaments produced with experiment 1 methods (FIG. 2B) maintaining structural stability post-extrusion were no larger than 1.25 mm. This shows the potential of the experiment 2 method involving printing directly into tubes for producing filaments at least twice as thick as what was possible with chaotic printing to this point.

Cell Viability and Expansion in Experiment 2. Experiment 2 cell expansion results (Table 1) demonstrate that chaotically printed BM-hMSC-laden hydrogel filaments fit to our filament chamber design can survive for at least 1 week with as little as 2 flow-based media replacements per day. There were approximately 140% (1.4×) the number of cells in the static group filaments as the flow group filaments after 1 week of culture. However, the static group filaments were exposed to 14× the amount of nutrient media in 6-well plates (7 mL) as the flow group in bioreactor segments (0.5 mL). This difference was because 0.5 mL of media was required to fill the bioreactor segments of the flow group, whereas 7 mL was required for the cell-laden filaments in filament chambers to be fully submerged in each 3.5-cm-diameter well in the static group. Thus, the bioreactor design provides the means to maintain a comparable number of cells with the same volume of hydrogel and significantly less media than traditional well plates. A group of BM-hMSC-laden hydrogel filaments fit to filament chambers and left without any media replacements exhibited virtually no cell viability by day 4.

Current Challenges and Future Directions. While many obstacles were overcome in this example, some challenges remain that future work will address. One issue is maintaining sterility of the bioreactor system. Autoclaving the 1/16″ rubber tubing appeared to cause warpage that increased resistance to flow. Thus, an alternative sterilization method or choice of tubing may be necessary. It should also be noted that SA-GelMA hydrogels printed directly into filament chambers were not as consistently flush to the tube edges as purely SA hydrogels. This could be due to several factors, such as variation in flow profiles exiting the nozzle based on fluid properties, or complications due to non-crosslinked GelMA components exiting the nozzle and entering the tubes. We plan to optimize flow regimes to increase channel-bearing, filament-based BM-hMSC expansion and to determine whether an alternative SA-GelMA formulation will both improve stiffness (i.e., provide a hydrogel with stiffness that is conducive to higher cell expansion) and/or improve consistency of printing filaments flush to the filament chamber wall (i.e., via increased swelling). It is worth noting that 2% SA without cells has been proven to print flush to filament chambers consistently in this example and could be printed with cells in the future. In this example, BM-hMSCs have been maintained for a week in chaotically printed hydrogel filaments within tubular chambers under flow conditions, while a functional bioreactor system has been established that applies regulated flow and measures pH, O₂ concentration, and temperature. Our next studies will carefully control the flow rate and duration (i.e., more often during the day, continuous, or triggered by pH and O₂ sensors). On a spectrum of flow rate and frequency, too high a flow rate and/or frequency may kill cells from applied shear stress, whereas too low might deprive cells of nutrients and waste removal, also killing them. A future study can modulate flow rate and frequency to find an optimum combination for cell expansion rate and maximum yield within our bioreactor system. Maximum yield could then be correlated to cell number and density in hydrogel filament layers, as well as cell-laden and fugitive layer SAV. We will obtain these data by measuring cross-sectional images of chaotically printed filaments.

TABLE 1
Experiment 2 Cell Expansion.

| After 1 Week of Cell Expansion | Flow Group | Static Group |
|---|---|---|
| Cells per mL of Media | 765,265 | 75,209 |
| Percent Growth from Day 0 | 307% | 422% |
| Cells per Filament | 382,632 | 526,461 |

### CONCLUSION

The next generation of large-scale cell manufacturing systems will need to incorporate exponentially higher SAV to improve cell expansion rate and total yield. Achieving this result will increase the number of patients who can receive cell-based cancer and regenerative medicine therapies. Chaotic printing may be a biofabrication method capable of meeting this need by producing reliable, micro-to-nanoscale layered geometries within cell-laden hydrogel filaments. This study demonstrated the viability of clinically relevant BM-hMSCs in chaotically printed SA-GelMA hydrogel filaments, as well as the positive impact of the presence of open channels within filaments on cell expansion and total yield. Finally, a functional bioreactor system was established that can be used to optimize cell expansion and total yield in future work by optimizing nutrient media flow regimes.

### REFERENCES

[1] Lee, M. W.; et al. Mesenchymal stem cells in suppression or progression of hematologic malignancy: current status and challenges. Leukemia, 2019; 33 (3): 597-611.
[2] Couto, P. S.; et al. Expansion of human mesenchymal stem/stromal cells (hMSCs) in bioreactors using microcarriers: lessons learnt and what the future holds. Biotechnol. Adv., 2020; 45.
[3] Panchalingam, K. M.; et al. Bioprocessing strategies for the large-scale production of human mesenchymal stem cells: a review. Stem Cell Res. Ther., 2015; 6:225.
[4] Chávez-Madero, C.; et al. Using chaotic advection for facile high throughput fabrication of ordered multilayer micro- and nanostructure: continuous chaotic printing. Biofabrication, 2020; 12.
[5] Hobbs, D. M., Muzzio, F. J. The Kenics static mixer: a three-dimensional chaotic flow. Chem. Eng. J., 1997; 67 (3): 153-166.
[6] Ball, O; et al. 3D printed vascular networks enhance viability in high volume perfusion bioreactor. Ann. Biomed. Eng., 2016; 44 (2): 3435-3445.
[7] Mishra, R; et al. Growth factor dose tuning for bone progenitor cell proliferation and differentiation on resorbable poly(propylene fumarate) scaffolds. Tissue Eng. Part C Methods, 2016; 22 (9): 904-913.
[8] González Abrego, A. V. Design and fabrication of bioreactors for tissue engineering. MS Thesis, 2019; The Ohio State University, Columbus, OH.
[9] Bolivar-Monsalve, E. J.; et al. Continuous chaotic bioprinting of skeletal muscle-like constructs. Bioprinting, 2021; 21.

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for the preparation of a perfusable scaffold for cell culture, the method comprising:
providing a bioink composition and a fugitive ink composition;
chaotic printing the bioink composition and the fugitive ink composition to generate a microstructured precursor comprising a plurality of lamellar structures formed from the bioink composition;
curing the bioink composition to form a cured scaffold precursor; and
removing the fugitive ink composition from the cured scaffold precursor, thereby forming the perfusable scaffold;
wherein the fugitive ink composition comprises hydroxyethyl cellulose (HEC).

2. The method of claim 1, wherein the method further comprises dispersing a population of cells in the bioink composition prior to the chaotic printing.

3. The method of claim 1, wherein the method further comprises seeding the perfusable scaffold with a population of cells.

4. The method of any of claims 2-3, wherein the cells comprise pluripotent stem cells, multipotent stem cells, progenitor cells, terminally differentiated cells, endothelial cells, endothelial progenitor cells, immortalized cell lines, primary cells, or any combination thereof.

5. The method of any of claims 1-4, wherein chaotic printing of the bioink composition and the fugitive ink composition comprises inducing laminar flow of the bioink composition and the fugitive ink composition through a mixer that chaotically mixes the bioink composition and the fugitive ink composition to form lamellar interfaces between the bioink composition and the fugitive ink composition.

6. The method of any of claims 1-5, wherein chaotic printing of the bioink composition and the fugitive ink composition comprises coextruding the bioink composition and the fugitive ink composition through a mixer that chaotically mixes the bioink composition and the fugitive ink composition to form lamellar interfaces between the bioink composition and the fugitive ink composition.

7. The method of any of claims 5-6, wherein the mixer comprises a static mixer, such as a Kenics static mixer.

8. The method of any of claims 1-7, wherein the perfusable scaffold an average striation thickness of from 10 nm to 500 μm.

9. The method of any of claims 1-8, wherein the perfusable scaffold exhibits a surface-area-to-volume (SAV) of from 400 m⁻¹ to 5000 m⁻¹.

10. The method of any of claims 1-9, wherein the perfusable scaffold exhibits a surface density of at least 0.05 m² cm⁻³.

11. The method of any of claims 1-10, wherein the perfusable scaffold is produced in the form of a fiber.

12. The method of any of claims 1-11, further chaotic printing the bioink composition and the fugitive ink composition comprises 3D printing, electrospinning, extrusion, or any combination thereof.

13. The method of any of claims 1-12, wherein the bioink composition comprises a polymer.

14. The method of claim 13, wherein the polymer comprises a hydrogel-forming agent.

15. The method of any of claims 13-14, wherein the polymer comprises a polysaccharide, such as alginate, hyaluronic acid, agarose, or any combination thereof.

16. The method of any of claims 13-15, wherein the polymer comprises a protein or peptide, such as gelatin, collagen, or any combination thereof.

17. The method of any of claims 13-16, wherein the polymer comprises a synthetic polymer, such as a polyester (e.g., poly(propylene fumarate) (PPF), polycaprolactone, poly(lactic-co-glycolic acid), polylactic acid, polyglycolic acid, or any combination thereof).

18. The method of any of claims 13-17, wherein the polymer is crosslinkable.

19. The method of any of claims 13-18, wherein the polymer is present in an amount of from 0.5% to 20% by weight, based on the total weight of the bioink composition.

20. The method of any of claims 1-19, wherein the bioink composition comprises a bioactive agent, such as a growth factor, growth inhibitor, cytokine, steroid, antibiotic, morphogen, or any combination thereof.

21. The method of claim 20, wherein the bioink composition comprises a polymer and wherein the bioactive agent is conjugated to the polymer.

22. The method of claim 20, wherein the bioink composition comprises a population of nanoparticles, a population of microparticles, or any combination thereof, and wherein the bioactive agent is conjugated to the particles.

23. The method of claim 20, wherein the bioink composition comprises a population of nanoparticles, a population of microparticles, or any combination thereof, and wherein the bioactive agent is encapsulated or dispersed in the particles.

24. The method of any of claims 1-23, wherein the fugitive ink composition comprises a polymer.

25. The method of claim 24, wherein the polymer comprises a poly(alkylene oxide) block copolymer, such as a polyoxyethylene-polyoxypropylene (PEO-PPO) block copolymers (e.g., a poloxamer).

26. The method of any of claims 24-25, wherein the polymer is present in an amount of from 0.5% to 20% by weight, based on the total weight of the fugitive ink composition.

27. A method for the preparation of a perfusable scaffold for cell culture disposed within a housing, the method comprising:
providing a bioink composition and a fugitive ink composition;
chaotic printing the bioink composition and the fugitive ink composition into a housing comprising a fluid inlet, a fluid outlet, and a path for fluid flow from the fluid inlet to the to generate a microstructured precursor occupying the path for fluid flow, the microstructured precursor comprising a plurality of lamellar structures formed from the bioink composition;
curing the bioink composition to form a cured scaffold precursor occupying the path for fluid flow; and
removing the fugitive ink composition from the cured scaffold precursor, thereby forming the perfusable scaffold disposed within the housing.

28. The method of claim 27, wherein the method further comprises dispersing a population of cells in the bioink composition prior to the chaotic printing.

29. The method of claim 27, wherein the method further comprises seeding the perfusable scaffold with a population of cells.

30. The method of any of claims 27-29, wherein the cells comprise pluripotent stem cells, multipotent stem cells, progenitor cells, terminally differentiated cells, endothelial cells, endothelial progenitor cells, immortalized cell lines, primary cells, or any combination thereof.

31. The method of any of claim 27-30, wherein chaotic printing of the bioink composition and the fugitive ink composition comprises inducing laminar flow of the bioink composition and the fugitive ink composition through a mixer that chaotically mixes the bioink composition and the fugitive ink composition to form lamellar interfaces between the bioink composition and the fugitive ink composition.

32. The method of any of claims 27-31, wherein chaotic printing of the bioink composition and the fugitive ink composition comprises coextruding the bioink composition and the fugitive ink composition through a mixer that chaotically mixes the bioink composition and the fugitive ink composition to form lamellar interfaces between the bioink composition and the fugitive ink composition.

33. The method of any of claims 31-32, wherein the mixer comprises a static mixer, such as a Kenics static mixer.

34. The method of any of claims 32-33, wherein the chaotic printing of the bioink composition and the fugitive ink composition comprises coextruding the bioink composition and the fugitive ink composition with a crosslinking agent.

35. The method of claim 34, wherein the bioink composition comprises an alginate and wherein the crosslinking agent comprises a divalent cation.

36. The method of claim 35, wherein the crosslinking agent comprises a calcium salt such as calcium chloride.

37. The method of any of claims 27-36, wherein the perfusable scaffold an average striation thickness of from 10 nm to 500 μm.

38. The method of any of claims 27-37, wherein the perfusable scaffold exhibits a surface-area-to-volume (SAV) of from 400 m⁻¹ to 5000 m⁻¹.

39. The method of any of claims 27-38, wherein the perfusable scaffold exhibits a surface density of at least 0.05 m² cm⁻³.

40. The method of any of claims 27-39, wherein the perfusable scaffold is produced in the form of a fiber.

41. The method of any of claims 27-40, further chaotic printing the bioink composition and the fugitive ink composition comprises 3D printing, electrospinning, extrusion, or any combination thereof.

42. The method of any of claims 27-41, wherein the bioink composition comprises a polymer.

43. The method of claim 42, wherein the polymer comprises a hydrogel-forming agent.

44. The method of any of claims 42-43, wherein the polymer comprises a polysaccharide, such as alginate, hyaluronic acid, agarose, or any combination thereof.

45. The method of any of claims 42-44, wherein the polymer comprises a protein or peptide, such as gelatin, collagen, or any combination thereof.

46. The method of any of claims 42-45, wherein the polymer comprises a synthetic polymer, such as a polyester (e.g., poly(propylene fumarate) (PPF), polycaprolactone, poly(lactic-co-glycolic acid), polylactic acid, polyglycolic acid, or any combination thereof).

47. The method of any of claims 42-46, wherein the polymer is crosslinkable.

48. The method of any of claims 42-47, wherein the polymer is present in an amount of from 0.5% to 20% by weight, based on the total weight of the bioink composition.

49. The method of any of claims 27-48, wherein the bioink composition comprises a bioactive agent, such as a growth factor, growth inhibitor, cytokine, steroid, antibiotic, morphogen, or any combination thereof.

50. The method of claim 49, wherein the bioink composition comprises a polymer and wherein the bioactive agent is conjugated to the polymer.

51. The method of claim 49, wherein the bioink composition comprises a population of nanoparticles, a population of microparticles, or any combination thereof, and wherein the bioactive agent is conjugated to the particles.

52. The method of claim 49, wherein the bioink composition comprises a population of nanoparticles, a population of microparticles, or any combination thereof, and wherein the bioactive agent is encapsulated or dispersed in the particles.

53. The method of any of claims 27-52, wherein the fugitive ink composition comprises a polymer.

54. The method of claim 53, wherein the polymer comprises a poly(alkylene oxide) block copolymer, such as a polyoxyethylene-polyoxypropylene (PEO-PPO) block copolymers (e.g., a poloxamer).

55. The method of claim 53, wherein the polymer comprises hydroxyethyl cellulose (HEC).

56. The method of any of claims 54-55, wherein the polymer is present in an amount of from 0.5% to 20% by weight, based on the total weight of the fugitive ink composition.

57. A perfusable scaffold for cell culture prepared by the method of any of claims 1-56.

58. A bioreactor comprising a plurality of perfusable scaffolds, each prepared by the method of any of claims 1-56.

59. The bioreactor of claim 58, wherein each of the plurality of perfusable scaffolds is operatively coupled to a proximal collar and a distal collar.

60. The bioreactor of claim 59, wherein the bioreactor further comprises a first single input plate operatively coupled to each of the proximal collars, and a second single input plate operatively coupled to each of the distal collars.

61. The bioreactor of claim 60, wherein the first single input plate and the second single input plate are configured to apply mechanical stimulation to the plurality of perfusable scaffolds.

62. The bioreactor of any of claims 60-61, wherein the first single input plate and the second single input plate are configured to apply electrical stimulation to the plurality of perfusable scaffolds.

63. The bioreactor of any of claims 58-62, wherein the bioreactor further comprises a pH monitoring and control system, a temperature monitoring and control system, an O2 monitoring and control system, a CO₂ monitoring and control system, a glucose monitoring and control system, a lactate monitoring and control system, a fluid flow monitoring and control system, or any combination thereof.
